(19) United States
(12) Patent Application Publication
(10) Pub. No.: US 2014/0309578 A1
(43) Pub. Date: Oct. 16, 2014

(54) BIOMEDICAL IMAGING AND THERAPY USING RED BLOOD CELLS

(71) Applicant: Bahman Anvari, Tustin, CA (US)

(72) Inventor: Bahman Anvari, Tustin, CA (US)

(21) Appl. No.: 13/864,103

(22) Filed: Apr. 16, 2013

(51) Int. Cl. A61K 47/46; A61K 41/00; A61N 5/06

(52) U.S. Cl. CPC A61K 47/46; A61N 5/062; A61K 41/0057
USPC 604/20

(57) ABSTRACT

Certain embodiments of the present invention provide methods, of treating a skin abnormality in a mammalian subject, that involve introducing, into a vasculature of the subject, red blood cells (RBCs) that comprise a photosensitive compound; and then permitting to pass a time-period sufficient for some of the RBCs to enter a region of the subject that comprises the skin abnormality; and then exposing RBCs in the region to an amount of radiation energy sufficient to result in the photosensitive compound mediating a hyperthermic therapy, a thermal therapy, an oxygen singlet therapy, a radical molecule therapy, or a combination thereof on the skin abnormality. In some embodiments, the photosensitive compound comprises a dye and is substantially encapsulated within the RBCs. In some embodiments, the radiation energy consists essentially of radiation wavelengths absorbed substantially more efficiently by the photosensitive compound than by an epidermal tissue of the subject.

### PRIORITY DATA

The present application claims the benefit of U.S. Provisional Patent Application Ser. No. 61/625616, filed Apr. 17, 2012.

### FIELD OF THE INVENTIONS

Certain embodiments of the invention relate to compositions that comprise red blood cells (RBCs) loaded with agents, the compositions having biomedical imaging, diagnostic, and therapeutic applications. Certain embodiments of the invention relate to methods of using agent-loaded RBC compositions in biomedical imaging, diagnostic, and/or treatment applications.

### BACKGROUND OF THE INVENTIONS

Erythrocytes, or RBCs, constitute the largest population of blood cells and are the main carriers of oxygen to the body's cells and tissues. RBCs are components that make up blood plasma along with platelets, leukocytes, salts, and proteins. RBCs contain high concentrations of iron-rich hemoglobin, and they can be easily isolated through centrifugation and other techniques. In the human body, the mature RBC is normally a non-nucleated, yellowish, biconcave disk with a central pallor. The biconcave shape provides a large surface-to-volume ratio and flexibility in narrow capillaries. (Millan et al., Journal of Controlled Release. 95:27 (2004)).

RBCs, despite their large diameters and volumes, readily conform to small capillary diameters, and have been demonstrated to possess properties that make them useful as carriers of molecules other than hemoglobin. RBCs are capable of reversible deformation, such as occurs when they are in hypotonic solution: their volumes increase causing 200-500 Å pores to open in their extracellular membranes and allowing two-way, trans-membrane exchange between their normal content (e.g., hemoglobin) and low to high molecular-weight substances placed in their externally vicinity. Then, by returning the solution to physiologic tonicity, the pores close and the cells return to normal size, trapping the added substances inside. Remaining non-entrapped substance can be washed away, leaving substance-loaded, osmotically-competent RBCs. Substance-loaded RBCs appear to have a normal life span of up to 120 days. (Seeman, J Cell Biology. 32:55 (1967) and USPAP 2011/0041133, the entire contents of each of which are hereby incorporated by reference).

### SUMMARY OF THE INVENTIONS

Agent-loaded RBCs of the invention provide a platform for multi-functional optical imaging through various modalities (e.g., fluorescence and photoacoustic imaging) as well and therapy through various mechanisms (e.g., drug delivery, photothermal, and photodynamic therapy). The surface of agent-loaded RBCs can be utilized to present an array of targeting moieties, such that agent-loaded RBCs that comprise a targeting moiety can localize to molecular biomarkers of various pathological and physiological conditions (e.g., a variety of tumors and/or cancers and skin abnormalities). Agent-loaded RBCs may be used for both optical imaging and phototherapy for these conditions and others.

Certain embodiments of the invention provide methods of treating a skin abnormality in a subject. The methods involve: introducing, into a vasculature of the subject, RBCs that comprise a photosensitive compound; permitting to pass, after introducing the RBCs into the vasculature, a time-period sufficient to allow a portion of the RBCs to enter a region of the subject in proximity with the skin abnormality; and exposing, on one or more occasions within 100 days, 90 days, 80 days, 70 days, 60 days, 50 days, 40 days, or 30 days after introducing the RBCs into the vasculature, at least a portion of the RBCs in the region to an amount of radiation energy effective to activate the photosensitive compound to mediate a function on the skin abnormality selected from hyperthermic therapy and oxygen singlet therapy, thereby treating the skin abnormality. In some embodiments, at least a portion of the skin abnormality is present in at least one of an epidermal region and a dermal region of the subject. In some embodiments, the photosensitive compound is substantially encapsulated within the RBCs. In some embodiments, the photosensitive compound comprises a dye (e.g., indocyanine green (ICG)). In some embodiments, the radiation energy consists essentially of at least one radiation wavelength that is absorbed substantially more efficiently by the photosensitive compound than by an epidermal tissue or a dermal tissue of the subject. In some embodiments, the skin abnormality is a port wine stain, a birthmark, a hemangioma, a mole, a melanoma, or a combination thereof. In some embodiments, the radiation energy consists essentially of radiation having wavelengths between about 700 nanometers and about 850 nanometers.

Certain embodiments of the invention provide methods of imaging a tumor or a cancer in a subject. The methods involve introducing, into a vasculature of the subject, RBCs that comprise a targeting moiety and a photosensitive compound that comprises an ICG; permitting to pass, after introducing the RBCs into the vasculature, a time-period sufficient to allow a portion of the RBCs to localize to cells of the tumor or the cancer; exposing the localized RBCs to an amount of radiation energy sufficient to result in the ICG generating fluorescence, heat, or both in amounts effective to mediate, on at least a portion of the tumor or the cancer, an imaging technique selected from fluorescent imaging and photoacoustic imaging; and performing the imaging technique. In some embodiments, the targeting moiety is present on or near an extracellular membrane of the RBC and comprises an antibody directed against an epitope present on or near cells of the tumor or the cancer. In some embodiments, the photosensitive compound is substantially encapsulated with the RBC. In some embodiments, the radiation energy consists essentially of radiation having wavelengths of about 650 nm to about 850 nm. In some embodiments, the imaging technique is performed on one or more occasions within 100 days, 90 days, 80 days, 70 days, 60 days, 50 days, 40 days, or 30 days after introducing the RBCs into the vasculature.

Certain embodiments of the invention provide methods treating a tumor in a mammalian subject. The methods comprise introducing, into a vasculature of the subject, RBCs that comprise a targeting moiety and an ICG; and then permitting to pass a time-period sufficient for a portion of the RBCs to localize to the tumor; and then exposing at least some of the localized RBCs to an amount of radiation energy sufficient to result in the ICG generating heat in amounts sufficient to result in at least some of the tumor being damaged or destroyed. In some embodiments, the targeting moiety is coupled to extracellular surfaces of the RBCs and comprises an antibody that binds an epitope present on or near cells of the tumor. In some embodiments, the ICG is substantially encapsulated within the RBCs. In some embodiments, the radiation energy consists essentially of radiation having wavelengths of about 650 nm to about 800 nm.

### DETAILED DESCRIPTION OF THE INVENTIONS

Certain embodiments of the present invention provide compositions that comprise RBCs loaded with agents and/or compounds, the compositions having biomedical imaging, diagnostic, and/or therapeutic applications. In some embodiments, the RBCs comprise one or more targeting moieties operative to localize the RBCs to a particular type of tissue or cell, including a normal tissue or cell; an aberrant tissue or cell (e.g., a tumor or cancer cell); an agent-loaded RBC of the invention; and combinations thereof. In some embodiments, a RBC of the invention comprises a tissue and/or cell targeting moiety positioned on or near an extracellular membrane of the RBC. As used herein, the term “targeting moiety” includes any compound, molecule, polymer, etc., be it small, macro, chemical, biological, etc. that comprises one or more chemical or functional group(s) operative to bind to a targeted tissue, cell, or other biologic structure, and thereby localize the targeting moiety (and any compound, molecule, polymer, cell, etc. to which the targeting moiety is coupled) to the targeted tissue, cell, or other biological structure. Agents and/or compounds loaded into RBCs of the invention include at least one of a small molecule, a dye, polymer, a peptide, a protein, a nucleic acid sequence, a salt, an acid, a base, and a buffer. In some embodiments, an agent and/or compound loaded into an RBC of the invention is formulated in a manner that substantially reduces or eliminates one or more of its physiological and/or chemical functions for a period of time or under certain conditions.

Non-limiting examples of tumor and/or cancers that may be imaged or treated with agent-loaded RBCs of the invention include those arising from and/or afflicting tissues of the breast, lung, stomach, ovary, prostate, liver, pancreas, and colon.

In certain embodiments, RBCs of the invention comprise at least one targeting moiety and at least one agent. In some embodiments, the at least one targeting moiety is present on or near an extracellular surface of the RBCs and the at least one agent is encapsulated within the extracellular membranes of the RBCs. In some embodiments, the targeting moiety is operative, following introduction of the RBCs into a subject's circulatory system, to localize at least some of the RBCs to particular cell or tissue types in the subject. In some embodiments, the at least one encapsulated agent is a photosensitive compound that efficiently absorbs radiation energy of a particular wavelength or range of wavelengths and, in response, fluoresces, heats, and/or generates oxygen singlets. In some embodiments, radiation wavelengths efficiently absorbed by a photosensitive compound are absorbed substantially less efficiently by cells or tissues of a subject exposed thereto.

Photosensitive compounds useful in certain embodiments of the invention include fluorescent dyes, non-limiting examples of which are ICG, fluorescein, rose bengal, IR700, IR 780, IR 783, dye 800, squaraine derivatives, phthalocyanine derivatives, BODIPY, Cy3, Cy5, Cy 7 and analogue members of the cyanine and tricarbocyanine dyes. ICG is commercially available and FDA-approved for administration to humans under several indications.

Hence, in some embodiments, a population of RBCs of the invention that comprise a targeting moiety and encapsulate the photosensitive compound, ICG, may be introduced into the circulatory system of a subject. Following such introduction, at least a portion of the RBC population localizes to cell and/or tissue types recognized by the targeting moiety and a portion of the RBC population travels throughout the circulatory system. Exposure of the localized RBC population to operative amounts of 700-850 nm radiation (e.g., from an infra-red laser) allows for angiographic imaging, hyperthermic treatment, and/or oxygen singlet treatment of tissues and cells in and around the area of localization, with relatively low levels of damage to tissues or cells of the subject outside of the area of localization. Exposure of the circulating RBC population to operative amounts of 700-850 nm radiation at a selected location accessible to the RBCs allows for angiographic imaging, hyperthermic treatment, and/or oxygen singlet treatment of tissues of the subject at the selected location, with relatively low levels of damage to non-selected tissues or cells of the subject.

Methods for positioning a targeting moiety of the invention on or near the extracellular membranes of RBCs include those described herein, and methods for encapsulating agents of the invention within the extracellular membranes of RBCs invention include those described herein. Methods for introducing agent-loaded RBCs of the invention into the circulatory system of a subject include any route of administering to a subject such RBCs effective to enable the RBCs to perform their intended function, non-limiting examples of which are orally, intranasally, parenterally (intravenously, intramuscularly, intraperitoneally, or subcutaneously), rectally, and topically.

Radiation energy useful in the imaging, hyperthermic treatment, and oxygen singlet treatments methods of the invention may come from any suitable source, including a laser and a pulse laser, and may be transcutaneously applied to a subject or applied from a position within the body of a subject. The particular radiation energy source and amount of energy applied will depend upon the type of photosensitive compound (e.g., fluorescent dye) loaded into RBCs utilized in practicing methods of the invention, and photosensitive compounds can be selected to match specific wavelengths of laser energy. Exemplary lasers for use with ICG include GentIeLASE® (Candela Corporation) and the Odyssey® NAVIGATOR' Diode laser (Ivoclar/Vivodent, Inc.).

In certain embodiments, RBCs of the invention comprise at least one targeting moiety on or near extracellular surfaces of the RBCs and a plurality of agents encapsulated within the extracellular membranes of the RBCs. In some embodiments, the targeting moiety is operative, following introduction of the RBCs into a subject's circulatory system, to localize at least a portion of the RBCs to cancer cells or tumor cells in the subject. In some embodiments, the targeting moiety comprises an antibody directed against a tumor-specific epitope. In some embodiments, the plurality of agents comprises a photosensitive compound and one or more chemotherapeutic compound(s). In some embodiments, the photosensitive compound is ICG and the chemotherapeutic compound is one or a combination of an amatoxin, an anthracycline, a vinca alkaloid, an anti-tubulin drug, an or an alkylating agent. Representative specific chemotherapeutic compounds include cisplatinum, adriamycin, dactinomycin, mitomycin, caminomycin, daunomycin, doxorubicin, tamoxifen, taxol, taxotere, vincristine, vinblastine, vinorelbine, etoposide, 5-fluorouracil, cytosine arabinoside, cyclophosphamide, thiotepa, methotrexate, camptothecin, actinomycin-D, mitomycin C, aminopterin, alpha-amanatin, combretastatin(s), and derivatives and prodrugs thereof.

Hence, in some embodiments, a population of RBCs of the invention that comprise a cancer-cell or tumor-cell targeting moiety and encapsulate ICG and one or more chemotherapeutic compounds may be introduced into the circulatory system of a subject that has a cancer or tumor. Following such introduction, at least a portion of the RBC population localizes to cancer or tumor cells recognized by the targeting moiety and a portion of the RBC population travels throughout the circulatory system. Exposure of the localized RBC population to operative amounts of 700-850 nm radiation (e.g., from an infra-red laser) allows for angiographic imaging, hyperthermic treatment, and/or oxygen singlet treatment of cancer or tumor cells at which the RBCs localize, with relatively low levels of damage to tissues or cells of the subject outside of the area of localization. Hyperthermic treatment may be achieved by exposing ICG encapsulated within the localized RBC population to amounts of 700-850 nm radiation that result in ICG mediated heating effective to lyse the encapsulating RBCs and damage or destroy at least some of the cancer or tumor cells at which the RBCs are localized. Such RBC lysis also releases the chemotherapeutic compound(s) encapsulated therein, and can deliver therapeutically effective concentrations of the chemotherapeutic compounds to at least some of the cancer or tumor cells at which the lysed RBCs were localized.

In certain embodiments, a second population of agent-loaded RBCs of the invention may comprise at least one targeting moiety on or near extracellular surfaces of the second population of RBCs operative to localize at least a portion of the second population of RBCs to at least a portion of a first population of RBCs. In some embodiments, the targeting moiety on the second population is an antibody directed against an epitope present on or near the extracellular surfaces of the first population of RBCs. In some embodiments, the second population of RBCs may be introduced into the circulatory system of a subject after or contemporaneously with the first population of RBCs, and the second population of RBCs may co-localize with the first population of RBCs to the cells and tissues recognized by the targeting moiety of the first population of RBCs. Such co-localization may be effective to amplify the total number of RBCs of the invention localized at the tissues or cells recognized by the targeting moiety of the first population of RBCs.

Amounts of RBCs of the invention administered to a subject, amounts of targeting moieties present on or near extracellular membranes of the RBCs of the invention, and concentrations of agents and/or compounds encapsulated within RBCs of the invention may vary between applications, subjects, etc. Such amounts encompass any sufficient to achieve a purpose of the invention, and include the following without limitation. Ranges of RBCs of the invention introduced into a subject in a single administration include 1×10³ to 1×10¹⁰ RBCs; 1×10⁴ to 1×10⁹ RBCs; and 1×10⁵ to 1×10⁸ RBCs. Ranges of targeting moieties present on or near extracellular membranes of an RBC of the invention include 1 to 5,000,000 molecules, 10 to 1,000,000 molecules, 100 to 500,000 molecules, 100 to 500,000 molecules, and 100 to 100,000 molecules. Concentration ranges of agents and/or compounds encapsulated within an RBC of the invention include 0.001 μg/ml to 1000 μg/ml, 0.01 μg/ml to 500 μg/ml, 0.1 μg/ml to 500 μg/ml, 1 μg/ml-500 pg/ml, 1 μg/ml to 100 μg/ml, and 1 μg/ml to 10 μg/ml.

Amounts of radiation operative to achieve imaging, hyperthermic therapy, and oxygen singlet therapy with agent and/or compound loaded RBCs of the invention may vary between particular photosensitive compounds and concentrations thereof within an RBC of the invention, applications, subjects, etc. Such amounts encompass any sufficient to achieve a purpose of the invention. Ranges of radiation energy operative to achieve imaging applications of the invention with ICG include 0.001-10 J/cm², irradiation time between 1 ms-10 minutes, wavelength in the range of 650-800 nm. Ranges of radiation energy operative to achieve hyperthermic therapy and/or RBC heat-lysis with ICG include 10-1000 J/cm², irradiation time between 1 ms-5 minutes, wavelength in the range of 700-850 nm. Amount ranges of radiation energy operative to achieve oxygen singlet therapy with ICG 0.1-1000 J/cm², irradiation time between 1 min-10 min, wavelength in the range of 700-850 nm.

### Isolating RBCs.

RBCs for use in generating agent and/or compound loaded RBCs of the invention can be isolated from whole blood using several methods, including without limitation, by means of a cell washer, a continuous flow cell separator, density gradient separation, fluorescence-activated cell sorting (FACS), Miltenyi immunomagnetic depletion (MACS), or combinations thereof. (See, e.g., van der Berg et al., Clin. Chem. 33:1081-1082(1987); Bar-Zvi et al., J. Biol. Chem. 262:17719-17723 (1987); and Goodman et al., Exp. Biol. Med. 232:1470-1476 (2007), the contents of which are hereby incorporated by reference in their entirety.)

In some embodiments, RBCs are isolated from whole blood by simple centrifugation. (See, e.g., van der Berg et al., Clin. Chem. 33:1081-1082 (1987).) For example, EDTA-anticoagulated whole blood may be centrifuged (e.g., at 850×g for 10 min at 4° C.) to separate platelet-rich plasma, buffy coat, and RBC components of whole blood. Then, plasma and buffy coat components are removed from the RBCs in the centrifuged whole blood sample, and the RBCs washed with isotonic saline solution (e.g., NaCl, 9 g/L).

In some embodiments, RBCs are isolated from whole blood by density gradient centrifugation with various separation mediums, such as Ficoll, Hypaque, Histopaque, Percoll, Sigmacell, or combinations thereof. For example, a volume of Histopaque-1077 may be layered on top of an equal volume of Histopaque-1119. Then, EDTA-anticoagulated whole blood, diluted 1:1 in an equal volume of isotonic saline solution (e.g., NaCl, 9 g/L), may be layered on top of the Histopaque and the sample centrifuged (e.g., at 700×g for 30 min at room temperature). In centrifugation, granulocytes migrate to the 1077/1119 interface; lymphocytes, other mononuclear cells, and platelets remain at the plasma/1077 interface; and RBCs are pelleted. The plasma and Hisopaque may then be removed from the RBC pellet, and RBCs washed with isotonic saline solution.

In some embodiments, RBCS may be isolated by centrifugation using a Percoll step gradient. (See, e.g., Bar-Zvi et al., J. Biol. Chem. 262:17719-17723 (1987).) For example, whole blood may be mixed with an anticoagulant solution (e.g., a solution containing 75 mM sodium citrate and 38 mM citric acid) and then washed with isotonic Hepes-buffered saline. Leukocytes and platelets may then be removed by adsorption on a-cellulose and Sigmacell (1:1). RBCs may be then be separated from reticulocytes and residual white blood cells by centrifugation through a 45/75% Percoll step gradient (e.g., at 2500 rpm for 10 min in a Sorvall SS34 rotor). In centrifugation, reticulocytes band at the 45/75% interface, remaining white blood cells band at the 0/45% interface, and RBCs are pelleted. The Percoll may be removed from the pelleted RBCs and the pelleted RBCs washed with isotonic Hepes-buffered saline.

In some embodiments, RBCs may be separated from reticulocytes using flow cytometry. (See, e.g., Goodman el al., Exp. Biol. Med. 232:1470-1476 (2007).) For example, whole blood may be centrifuged (e.g., at 550×g, 20 min, 25° C.) to separate cells from plasma. The resulting cell pellet may be resuspended in phosphate buffered saline solution and further fractionated on Ficoll-Paque (1.077 density) by centrifugation (e.g., at 400×g for 30 min, 25° C.) to separate the RBCs from white blood cells. The resulting cell pellet may be resuspended in, e.g., RPMI supplemented with 10% fetal bovine serum and sorted on a FACS instrument, such as a Becton Dickinson FACSCalibur (BD Biosciences, Franklin Lakes, N.J., USA) based on size and granularity.

In some embodiments, RBCs may be isolated by immunomagnetic depletion. (See, e.g., Goodman, el al., Exp. Biol. Med. 232:1470-1476 (2007).) For example, magnetic beads with cell-type specific antibodies may be used to eliminate non-RBCs from whole blood. In some embodiments, RBCs are isolated from whole blood using a density gradient protocol followed by immunomagnetic depletion of residual reticulocytes. The so-isolated RBCs may be pre-incubated with human antibody serum (e.g., for 20 min at 25° C.) and then incubated with antibodies directed against reticulocyte specific antigens, such as CD71 and CD36, the antibodies directly or indirectly attached to magnetic beads. Reticulocyte-antibody-magnetic bead complexes may then be selectively removed from RBCs by magnetic separation.

In certain embodiments, RBCs for use in generating agent-loaded RBCs of the invention may be derived from reticulocytes. In some embodiments, reticulocytes may be isolated from peripheral blood using differential centrifugation through density gradients, such as Percoll gradients. (See, e.g., Noble el al., Blood 74:475-481 (1989), the content of which is hereby incorporated by reference in its entirety.) For example, isotonic Percoll solutions (e.g., osmolarity between 295 and 310 mOsm) having a density of 1.096 or 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). Five milliliters of the first Percoll solution (density 1.096) is added to a sterile 15 ml conical centrifuge tube; two milliliters of the second Percoll solution (density 1.058) is layered over the higher-density, first Percoll solution; and two to four milliliters of whole blood is then layered on top of the second Percoll solution. The tube is centrifuged (e.g., at 250×g for 30 min in a refrigerated centrifuge with swing-out tube holders). In centrifugation, reticulocytes and some white cells migrate to the interface between the two Percoll layers. The cells at the interface are transferred to a new tube and washed with phosphate buffered saline (PBS) with 5 mM glucose, 0.03 mM sodium azide and 1 mg/ml BSA. Residual white blood cells may be removed by chromatography in PBS over a size exclusion column.

In some embodiments, reticulocytes may be isolated using an immunomagnetic separation approach. (See, e.g., Brun et al., Blood 76:2397-2403 (1990), the content of which is hereby incorporated by reference in its entirety.) For example, an antibody to the transferrin receptor may be used to selectively isolate reticulocytes from a mixed RBC population, due relatively high expression levels of the transferrin receptor by reticulocytes. The transferrin antibody (e.g., monoclonal antibody 10D2 against human transferrin) may be directly or indirectly linked to magnetic beads. The antibody and RBCs are incubated at 22° C. with gentle mixing for 60-90 min, followed by separation of the beads with attached reticulocytes using a magnetic field. The isolated reticulocytes may be removed from the magnetic beads using, for example, DETACHaBEAD® solution.

In some embodiments, agent-loaded RBCs of the invention may comprise isolated reticulocytes. In some embodiments, agent-loaded RBCs of the invention may comprise RBCs matured and/or differentiated from isolated reticulocytes. In some embodiments, maturation of reticulocytes may be carried out in vitro using standard cell culture methods. (See, e.g., Noble et al., Blood 74:475-481 (1998).)

In some embodiments, RBCs and/or reticulocytes for use in generating agent-loaded RBCs of the invention may be derived from hematopoietic stem cells isolated from bone marrow, umbilical chord blood, or normal peripheral blood following pre-treatment with cytokines, such as granulocyte colony stimulating factor, which mobilizes release of hematopoietic stem cells from the bone marrow compartment into the peripheral circulation. Such hematopoietic stem cells may be expanded and differentiated ex vivo into mature erythrocytes using standard methods. (See, e.g., Giarratana et al., Nature Biotech. 23:69-74 (2005), the entire content of which is hereby incorporated by reference in its entirety.)

In certain embodiment, agent-loaded RBCs of the invention are autologous to the subject. In certain embodiments, agent-loaded RBCs of the invention are allogenic to the subject. In some embodiments, ABO blood group compatibility between RBC donor and recipient are achieved in order to avoid an acute intravascular hemolytic transfusion reaction. Here, it is known that ABO blood types are defined based on the presence or absence, on the surface of RBCs, of monosaccharide carbohydrate antigens A and B. (See, e.g., Liu et al., Nat. Biotech. 25:454-464 (2007), the entire content of which is hereby incorporated by reference in its entirety.) Individuals with group A RBCs usually have antibodies directed against group B red blood cells, and vice versa. Individuals with group AB RBCs usually have neither antibody, and individuals with blood group O RBCs usually have both antibodies. Individuals with either anti-A and/or anti-B antibodies should not receive a transfusion of RBCs that comprise the corresponding antigen. Because group O RBCs contain neither A nor B antigens, they can be transfused into recipients of any ABO blood group type.

In certain embodiments, non-group O RBCs may be modified to the group O type. For example, enzymatic removal of antigen-A and antigen-B monosaccharides on the surface of group A, group B, and group AB RBCs may be performed according to standard methods to generate group O-like RBCs. (See, e.g., Liu et al., Nat. Biotech. 25:454-464 (2007).)

### Targeting Moieties.

In certain embodiments, agent-loaded RBCs of the invention comprise a targeting moiety operative to localize RBCs at particular cells, tissues, receptors, or subsets thereof, present in the body of a subject. Examples of targeting moieties include, without limitation, biotin; avidin; streptavidin; folate, ligand; receptor; polymer; carbohydrate; oligosaccharide; polysaccharide; DNA; RNA; aptamer; peptide; protein; lectin; lipid; and antibody.

Antibodies useful as targeting moieties in certain embodiments of the invention include monoclonal and polyclonal antibodies, and functional fragments or derivatives thereof, such as Fabs and scFvs. Such antibodies specifically bind to at least one epitope of a molecule associated with, or on the surface of, a target cell or tissue. And such antibodies may be univalent or multivalent, as well as monospecific or multispecific. By “multivalent” it is meant that an antibody may simultaneously bind more than one epitope, which may have the same or a different structure. By “multispecific” it is meant that an antibody may bind two or more epitopes having different structure. Accordingly, an antibody that recognizes two different epitopes would be considered multivalent and multispecific. Antibodies for use in subjects include those are those that are substantially or entirely non-immunogenic when administered to the subject, and when intended for use in human subjects and originating from non-human animals, are commonly referred to as “humanized,” “human,” “chimeric,” or “primatized” antibodies.

Antibodies useful as targeting moieties in certain embodiments of the invention include those directed against epitopes present at the extracellular surface of tumor or cancer cells. In some embodiments, such epitopes absent from the extracellular surface of non-tumor or non-cancer cells, or present on a limited set of non-tumor or non-cancer cell types. In some embodiments, such epitopes are present at the surface of cancer or tumor cells at higher levels than on non-tumor or non-cancer cells. Antibodies useful in certain embodiments of the invention include antibodies directed to receptor tyrosine kinase proteins, such as members of the EGF, FGF, VEGF, PDGF, insulin, and HGF recptor tyrosine kinase families. Antibodies useful in certain embodiments of the present invention include antibodies directed against MUC1; antibodies directed against SIMA135; antibodies directed against Lewis antigens; antibodies directed against CD20, such as Rituximab (Genentech), tositumomab (Corixa/GSK), ofatumumab (Genmab), and ibritumomab (Biogen Idec); antibodies directed against CD30, such as brentuximab (Seattle Genetics); antibodies directed against HER2, such as trastuzumab (Genentech), pertuzumab (Genentech), and mAB 7.16.4 (U.S. Pat. No. 5,705,157); antibodies directed against CD33, such as gemtuzumab (Wyeth/Pfizer); antibodies directed against CD52, such as alemtuzumab (Genzyme); antibodies directed against EGFR, such as cetuximab (ImClone/Lilly) and panitumumab (Amgen); antibodies directed against VEGF, such as bevacizumab (Genentech); and antibodies directed against CTLA-4, such as ipilimumab (BMS).

Polymers useful as targeting moieties in certain embodiments of the invention include those that are insoluble above a pH or pH range and soluble below that pH or pH range. Examples of such polymers include polymers of 4-amino-N-[4,6-dimethyl-2-pyrimidinyl]benzene sulfonamide derivatized with N,N-dimethylacrylamide; polymers of polyacrylamide, polymers of chitosan, and polymers of dendrimer.

Targeting moieties may be coupled to RBCs of the invention in a variety of ways, before or after RBCs are loaded with agents and/or compounds. In some embodiments, such coupling may be achieved as a result of a targeting antibody being part of a multispecific antibody complex composed of at least one component that recognizes a specific epitope on the surface of the target cell or tissue and at least one component that recognizes an epitope on the surface of RBCs. Such RBC epitopes include, without limitation, α-N-acetylgalactosaminyltransferase, complement C4, aquaporin, complement decay-accelerating factor, band3 anion transport protein, Duffy antigen, glycophorin A, B and/or C, galactoside 2-L-fucosyltransferase 1, galactoside 2-L-fucosyltransferase 2, galactoside 3(4)-L-fusosyltransferase, CD44, Kell blood group glycoprotein, urea transporter, complement receptor protein (CR1), membrane transport protein XK, Landsteiner-Wiener blood group glycoprotein, Lutheran blood group glycoprotein, blood group RH (CE) polypeptide, blood group RH (D) polypeptide, Xg glycoprotein, acetylcholinesterase, anion exchanger, and/or insulin receptor.

In some embodiments, two or more antibodies may be linked through disulfide bonds. For example, a targeting antibody is reacted with N-succinimidyl S-acetylthioacetate (SATA) and subsequently deprotected by treatment with hydroxylamine to generate an SH-antibody with free sulfhydryl groups. The RBC-epitope binding antibody is reacted with sulfosuccinimidyl 4-(N-maelimidomethyl)cyclohexane-1-carboxylate (sSMCC). The two antibodies treated as such are purified by gel filtration and then reacted with one another to form a bispecific antibody complex.

In some embodiments, the antibodies may be chemically cross-linked to form a heteropolymerized complex using, for example, SPDP [N-succinimidyl-3-(2-pyridyldithio)propionate]. (See, e.g., Liu el al., Proc. Nat'l Acad. Sci. USA 82:8648-8652 (1985), the entire content of which is hereby incorporated by reference in its entirety.) To generate a complex, the targeting antibody (e.g., 1-2 mg/ml) is incubated with a 7-fold molar excess of SPDP in phosphate buffered saline (PBS) for 45 minutes at room temperature. Excess SPDP is removed by dialysis overnight against two changes of PBS. Thiol groups are attached to the RBC-epitope binding antibody by incubating the antibody (e.g., 1-3 mg/ml) with a 1000-fold molar excess of 2-iminothiolane in 12.5 mM sodium borate/PBS for 45 min at room temperature. Excess 2-iminothiolane is removed by dialysis as above. Equimolar amounts of the modified antibodies are incubated for 7 h at room temperature and the resulting heteropolymerized complex is separated from the uncoupled antibodies based on molecular weight using a standard sizing column.

In certain embodiments, a targeting moiety may be bound to the surface of a RBC of the invention through a biotin-streptavidin bridge. For example, a biotinylated antibody, peptide, protein, or other targeting moiety may be linked to a non-specifically biotinylated RBC surface through a streptavidin bridge. And such targeting moieties can be conjugated to biotin by a number of chemical methods. (See, e.g., Hirsch et al., Methods Mol. Biol. 295:135-154 (2004), the entire content of which is hereby incorporated by reference in its entirety.) RBC surface membrane proteins may be biotinylated using an amine reactive biotinylation reagent, such as EZ-Link Sulfo-NHS-SS-Biotin (sulfosuccinimidyl 2-(biotinamido)-ethyl-1,3-dithiopropionate. (See, e.g., Jaiswal et al., Nature Biotech. 21:47-51 (2003), the entire content of which is hereby incorporated by reference in its entirety.) RBCs may be incubated for 30 min at 4° C. in 1 mg/ml solution of sulfo-NHS-SS in phosphate-buffered saline. Excess biotin reagent is removed by washing the cells with Tris-buffered saline. The biotinylated cells are then reacted with the biotinylated targeting moiety in the presence of streptavidin to couple targeting moiety with RBC through a streptavidin bridge.

In certain embodiments, a targeting moiety may be coupled to a RBC via a covalent attachment. For example, a targeting moiety may be derivatized and bound to a RBC using a coupling compound containing an electrophilic group that will react with nucleophiles on a RBC to form covalent bond. Such electrophilic groups include, without limitation, α, β unsaturated carbonyls, alkyl halides and thiol reagents such as substituted maleimides. In addition, a coupling compound may be coupled to a targeting moiety via one or more of the functional groups in the targeting moiety, such as amino, carboxyl and tryosine groups. For this purpose, coupling compounds should contain free carboxyl groups, free amino groups, aromatic amino groups, or other groups capable of reaction with enzyme functional groups.

In certain embodiments, highly charged derivatives of a targeting moiety may be prepared for immobilization on RBCs through electrostatic bonding. Such derivatives include, without limitation, polylysyl and polyglutamyl enzymes. The choice of the reactive group embodied in the derivative depends on the reactive conditions employed to couple the electrophile with the nucleophilic groups on a RBC for immobilization. Such coupling immobilization reactions can proceed in a number of ways. Typically, a coupling agent can be used to form a bridge between a macromolecule, such as a polymer, and a RBC. In this case, the coupling agent should possess a functional group, such as a carboxyl group, that can be reacted with the targeting moiety. One pathway for preparing the macromolecular derivative comprises the utilization of carboxyl groups in the coupling agent to form mixed anhydrides which react with the target recognition moiety, in which use is made of an activator which is capable of forming the mixed anhydride. Representative of such activators are isobutylchloroformate or other chloroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the target recognition moiety to yield the reactive derivative which in turn can react with nucleophilic groups on the red blood cell to immobilize the macromolecule.

Functional groups on a targeting moiety, such as carboxyl groups, can be activated with carbodiimides and the like activators. Subsequently, functional groups on the bridging reagent, such as amino groups, can be reacted with the activated group on the targeting moiety to form the reactive derivative. In addition, the coupling agent should possess a second reactive grouping which will react with appropriate nucleophilic groups on RBCs to form the bridge. Typical of such reactive groupings are alkylating agents such as iodoacetic acid, α, β unsaturated carbonyl compounds, such as acrylic acid and the like, thiol reagents, such as mercurials, substituted maleimides and the like.

In certain embodiments, functional groups on a targeting moiety can be activated so as to react directly with nucleophiles on RBCs, which obviates need for a bridge-forming compound. For this purpose, beneficial use is made of an activator such as Woodward's Reagent K or the like which brings about the formation of carboxyl groups in the targeting moiety into enol esters, as distinguished from mixed anhydrides. The enol ester derivatives of targeting moieties will subsequently react with nucleophilic groups on RBCs to effect coupling of the macromolecule.

In certain embodiments, RBC precursors, such as reticulocytes and hematopoietic stem cells, may be genetically engineered to express one or more agents and/or targeting moieties of the invention. In some embodiments, isolated reticulocytes and/or stem cells may be transfected with mRNA or DNA expression constructs that encode nucleic acid, protein, and/or peptide agents and/or targeting moieties of the invention, resulting in the expression of such agents and/or targeting moieties on the cell surface or interior of transfected RBCs. Such RNA and DNA sequences may be introduced into reticulocytes using a variety of approaches including lipofection and electroporation. (van Tandeloo et al., Blood 98:49-56 (2001), the entire content of which is hereby incorporated by reference in its entirety.) For lipofection, e.g., 5 μg of in vitro transcribed mRNA is incubated for 5-15 min at a 1:4 ratio with the cationic lipid DMRIE-C. Alternatively, a variety of other cationic lipids or cationic polymers may be used to transfect cells with mRNA including, for example, DOTAP, and various forms of polyethylenimine, and polyL-lysine. (Bettinger et al., Nucleic Acids Res. 29:3882-3891 (2001), the entire content of which is hereby incorporated by reference in its entirety.) The resulting mRNA/lipid complexes are incubated with cells (e.g., 1-2×10⁶ cells/ml for 2 h at 37° C.), washed and returned to culture. For electroporation, e.g., 5-20×10⁶ cells are mixed with about 20 μg of in vitro transcribed mRNA and electroporated in a 0.4-cm cuvette. Various voltages, capacitances and electroporation volumes may be tested to determine the optimal transfection conditions of a particular mRNA into a reticulocyte.

In some embodiments, RBCs of the invention may be genetically engineered to express a cell membrane associated receptor, a cell membrane associated antibody, a ligand, a fluorescent protein, or chimeras or derivatives or mutants thereof.

### Loading RBCs.

In certain embodiments of the invention, RBCs are loaded with one or more agents, such that the agents are encapsulated within the RBC. A number of methods may be used to load modified RBCs with an agent, such as, without limitation, hypotonic dialysis, osmosis, osmotic pulsing, osmotic shock, ionophoresis, electroporation, sonication, microinjection, calcium precipitation, membrane intercalation, lipid mediated transfection, detergent treatment, viral infection, diffusion, use of protein transduction domains, particle firing, membrane fusion, freeze-thawing, mechanical disruption, and filtration. (See, e.g., U.S. Pat. No. 6,495,351 B2 and USPAP 2007/0243137).

In some embodiments, RBCs may be loaded with an agent using dialysis against a hypotonic solution to swell the cells and create pores in the cell membrane. (See, e.g., U.S. Pat. Nos. 4,327,710, 5,753,221, and 6,495,351.) For example, a pellet of isolated RBCs is resuspended in 10 mM HEPES, 140 mM NaCl, 5 mM glucose pH 7.4 and dialyzed against a low ionic strength buffer containing 10 mM NaH2PO4, 10 mM NaHCO3, 20 mM glucose, and 4 mM MgCl₂, pH 7.4. After 30-60 min, the RBCs are further dialyzed against 16 mM NaH₂PO₄, pH 7.4 solution containing an agent for an additional 30-60 min, which effectively loads the agent into the RBC. All of these procedures may be performed at a temperature of 4° C. Membranes of such agent-loaded RBCs may be resealed by gentle heating in the a physiological solution, such as 0.9% saline, phosphate buffered saline, Ringer's solution, cell culture medium, blood plasma or lymphatic fluid, for 1-2 min at a temperature of 60° C. Alternatively, the cells may be incubated at a temperature of 25-50° C. for 30 min to 4 h. (See, e.g., U.S. Patent Application 2007/0243137). Alternatively, the disrupted RBCs may be resealed by incubation in 5 mM adenine, 100 mM inosine, 2 mM ATP, 100 mM glucose, 100 mM Na-pyruvate, 4 mM MgCl₂, 194 mM NaCl, 1.6 M KCl, and 35 mM NaH₂PO₄, pH 7.4 at a temperature of 37° C. for 20-30 min. (See, e.g., U.S. Pat. No. 5,753,221.)

In some embodiments, RBCs may be loaded with an agent using electroporation. For example, RBCs are suspended in a physiological and electrically conductive media, such as platelet-free plasma, and agent is added. 0.2 to 1.0 ml of the mixture is placed in an electroporation cuvette and cooled on ice for 10 min. The cuvette is placed in an electroporation apparatus, in which the cells are electroporated with a single pulse of approximately 2.4 milliseconds in length and a field strength of approximately 2.0 kV/cm. Alternatively, double pulses of 2.2 kV delivered at 0.25 μF may be applied to achieve loading capacity. The cuvette is returned to the ice bath for 10-60 min and then placed in a 37° C. water bath to induce resealing of RBC membranes.

In some embodiments, RBCs may be loaded with an agent and/or a compound using sonication. For example, modified RBCs are exposed to high intensity sound waves, causing transient disruption of the cell membrane allowing therapeutic agent to diffuse into the cell.

In some embodiments, RBCs may be loaded with an agent using detergent treatment. For example, RBCs are treated with a mild detergent which transiently compromises the cell membrane by creating holes through which therapeutic agent may diffuse. After cells are loaded, the detergent is washed from the cells.

In some embodiments, RBCs may be loaded with an agent by fusing or conjugating the agent to proteins and/or peptides capable of crossing or translocating the plasma membrane. (See, e.g., USPAP 2002/0151004.) Examples of protein domains and sequences that are capable of translocating a cell membrane include, for example, sequences from the HIV-1-transactivating protein (TAT), the Drosophila Antennapedia homeodomain protein, the herpes simplex-1 virus VP22 protein, and transportin.

In some embodiments, RBCs may be loaded with an agent using mechanical firing. For example, RBCs may be bombarded with therapeutic agent attached to a heavy or charged particle, such as gold microcarriers, and are mechanically or electrically accelerated such that they traverse the cell membrane. Microparticle bombardment of this sort may be achieved using, for example, a Gene Gun.

In some embodiments, RBCs may be loaded with an agent using a vesicle. For example, vesicles are loaded with the agent during vesicle formation or using one or more method described herein. The loaded vesicles are then fused with the RBCs, and such fusion may be facilitated using various reagents.

In some embodiments, RBCs may be loaded with a therapeutic agent using filtration. For example, the modified RBC and therapeutic agent may be forced through a filter of pore size smaller than the RBC causing transient disruption of the cell membrane and allowing therapeutic agent to enter the cell.

In some embodiments, RBCs may be loaded with an agent using freeze thawing. For example, a pellet of RBCs (0.1-1.0 ml) is mixed with an equal volume of an isotonic solution (e.g., phosphate buffered saline) containing the agent. The RBCs are frozen by immersing the tube containing the cells and therapeutic agent into liquid nitrogen or an ethanol-dry ice slurry. The cells are then thawed in a 23° C. water bath and the cycle repeated if necessary to increase loading.

Agents and Compounds Loaded into RBCs.

Agents and/or compounds useful for loading into RBCs of the invention have a variety of identities and functions and include, without limtation small molecules, dyes, peptides, proteins, salts, acids, bases, and buffers. In some embodiments, an agent and/or compound loaded into an RBC of the invention is formulated in a manner that substantially reduces or eliminates one or more of the its physiological and/or chemical functions or effects for a period of time or under certain conditions.

In certain embodiments, a compound loaded into RBCs of the invention may be capable of endothermic reaction in which ambient heat is absorbed. Non-limiting examples of such endothermic compounds include ammonium chloride, ammonium nitrate, and potassium chloride, potassium chloride, barium hydroxide octahydrate, and ammonium thiocyanate.

In some embodiments, biodissolvable and/or biodegradable polymers may be used to coat or encapsulate endothermic compounds loaded into RBCs comprising a cell or tissue targeting moiety, such that a substantial amount of the coated or encapsulated endothermic agent does not undergo endothermic reaction in the RBCs for a period of time or under certain conditions (e.g., pH about 6.9). Upon administration to a subject, a population of such RBCs localizes to the target tissue or cell type and remains there for a period of time, or experiences a pH about 6.9, sufficient to allow the coating or encapsulating polymer to dissolve and/or degrade, which triggers endothermic reactions sufficient to cool cells or tissue in the area that the RBC population is localized.

Non-limiting examples of biodissolvable and/or biodegradable coating or encapsulating polymers include hydrophobic, polyester polymers such as poly (ε-caprolactone), poly(alkylene glycol adipate), poly(propylene glycol adipate), poly(butylene glycol adipate), and blends and copolymers thereof. Poly(caprolactone) polymers are commercially available under the trade names TONE™ Polyol and CAPA™ Polyol, respectively.

In certain embodiments, an agent coupled to and/or loaded into RBCs of the invention may be capable of lysing RBCs in a pH-dependent manner. In some embodiments, such pH-dependent, RBC-lysing agents are substantially inactive at normal physiological pH ranges (e.g., pH 7.1 to 7.4) and substantially active at lower pH ranges (e.g., pH<about 7). Non-limiting examples of pH-dependent, RBC-lysing agents include polymers of ethyl acrylic acid (PEAA); polymers of propyl acrylic acid (PPAA); polymers of butyl acrylic acid (PBAA); and combinations thereof. Methods for preparing such PEAA, PPAA, and PBAA polymers are described in USPAP 2001/0007666, the entire content of which is hereby incorporated by reference. In some embodiments, pH-dependent, RBC-lysing agents are, at a physiologically normal pH (e.g., pH 7.1 to 7.4) coupled to and/or loaded into RBCs of the invention comprising a cancer or tumor cell targeting moiety. Upon administration of such RBCs to a subject having a cancer or tumor recognized by the targeting moiety, a population of such RBCs localizes to the target cancer or tumor, the local environment of which is characterized by having a low pH and triggers RBC lysis in the area that the RBC population is localized.

### Photoacoustic Imaging.

In photoacoustic imaging, non-ionizing laser radiation is delivered into biological tissues. Some of the delivered energy is absorbed and converted into heat, leading to transient thermoelastic expansion and thus wideband (e.g. MHz) ultrasonic emission. The generated ultrasonic waves are then detected by ultrasonic transducers to form images. It is known that optical absorption is closely associated with physiological properties, such as hemoglobin concentration and oxygen saturation. As a result, the magnitude of the ultrasonic emission (i.e., photoacoustic signal), which is proportional to the local energy deposition, reveals physiologically specific optical absorption contrast. 2D or 3D images of the targeted areas can then be formed. The optical absorption in biological tissues can be due to endogenous molecules such as hemoglobin or melanin, or exogenously delivered contrast agents, such as fluorescent dyes like ICG. Photoacoustic imaging can be used in vivo for tumor angiogenesis monitoring, blood oxygenation mapping, functional brain imaging, and skin melanoma detection.

Photoacoustic imaging relies on optical absorption for it signals. When photons are absorbed, nonradiative de-excitation of the absorbed optical energy takes place with the release of localized heat. The local thermal expansion that results produces pressure transients. When illuminated with pulsed laser light, a tumor site by virtue of its higher absorption with respect to the healthy background tissue, due to antiogenesis, will act as a source of bipolar photoacoustic pulses. The ultrasound propagates with minimum distortion to the surface where it is detected using appropriate wideband detectors. The time of flight, amplitude, and peak-peak time of the bipolar photoacoustic pulse possess information regarding the location, absorption, and dimension of the source, thereby permitting a reconstruction of the tumor site. The technique combines the specificity and sensitivity of optical interactions with the high resolution of ultrasound imaging.

### Oxygen Singlet Therapy.

A photosensitive compound includes a chemical compound, such as a dye, that upon exposure to photoactivating radiation releases a singlet oxygen molecule. In some embodiments, a photosensitive compound itself, or another compound, is converted into a cytotoxic form, whereby target cells are killed or their proliferative potential diminished. Certain photosensitive compounds molecules become toxic when activated by light, for example by generating toxic species: e.g., oxidizing agents such as singlet oxygen or oxygen-derived free radicals, which are extremely destructive to cellular material and biomolecules such as lipids, proteins and nucleic acids. ICGs and porphyrins are examples of photosensitizing agents that act by generation of toxic oxygen species, the effects of which are useful in providing oxygen singlet therapies of the invention. A listing of representative photosensitive compounds may be found in Kreimer-Bimbaurn, Sem. Hematol. 26:157-73 (1989), the entire contents of which is hereby incorporated by reference.

### In Vivo Assays.

H1299 cells are a non-small cell lung carcinoma cell line derived from the lymph node that has a homozygous partial deletion of the p53 gene and does not express the tumor suppressor p53 protein, which in part accounts for their proliferative propensity. SKOV3 ovarian cancer cells express high levels of HER2 and do not express p53. (Tolmachev, F. et al., Eur. J. Nuc. Mol. Imaging 38: 531-539 (2010) and Vikhanskaya, F. et al. Nuc. Ac. Res. 22(6):1012-1017 (1994), the entire contents of each of which are hereby incorporated by reference in their entirety.) OVCAR3 ovarian cancer cells express low levels of HER2 and low levels of R743G mutant p53. (Delord, J. et al., Ann. Oncol. 16:1889-1897 (2005) and Yaginuma, Y. and Westphal, H., Cancer Res. 52(4):4196-4199 (1992), the entire contents of each of which are hereby incorporated by reference in their entirety.)

H1299, SKOV3, and OVCAR3 cells can be transiently or stably transfected with constructs engineered to express therein any one of a variety of p53 mutant proteins (e.g., temperature sensitive p53 proteins), either constitutively or inducibly. For instance, H1299, SKOV3, and OVCAR3 cell lines can be cultured in an effective cell culture medium, such as RPMI-1640 medium (L-glutamine, NaHCO3) supplemented with 10% fetal calf serum and 1% penicillin/streptomycin at 5% CO2 and 32 or 37° C. To form H1299, SKOV3, or OVCAR3 cells expressing temperature p53 variants (TSp53), the cells can be co-transfected with TSp53-pT-REx-DEST30 (prepared according to the instructions of manufacturer) for constitutive expression and, optionally, pcDNA6/TR repressing TSp53 expression (Invitrogen) in ratio 1:7 for inducible expression, using Lipofectamine 2000 (Invitrogen). Stable transfectants may be selected with 500.25 g/ml Geneticin sulfate G418 (Gibco) and 5.25 g/ml Blasticidin S HCl (Invitrogen). Inducible expression of TSp53 in suchy transfected cells may be achieved by adding tetracycline (1.25 g/ml). Exemplary TSp53 proteins include R175H, R248W, P96A, R110L, Y126C, C135G,38V,59V, I195T, Y205C, S215G, V216M, Y220C, P222L, Y234C, M237K, I254N, G266E, V272G, V274G, E285K, E286K,E286V, R337C, and L344R.

Mice can be challenged with H1299, SKOV3, and OVCAR3 cells expressing a TSp53 protein (e.g., by transplantation into a tissue, injection into a vasculature, or topical application or injection into a peritoneal cavity of the mice) in amounts sufficient to allow the H1299, SKOV3, and OVCAR3 TSp53 expressing cells to establish one or more xenograft tumors in the mice. Following such challenge, agent-loaded RBCs and mock RBCs (e.g., RBCs lacking on or more of the agents loaded into an agent-loaded RBC) may be administered into the tissue, vasculature, or peritoneal cavity of so-challenged mice (typically groups of 3-10 mice are treated with agent-loaded RBCs or mock RBCs). And the agent-loaded RBCs' effect on H1299, SKOV3, and OVCAR3 TSp53 xenograft tumor establishment, growth, metastasis, etc. determined by standard assays and statistical methods. (See, e.g., Auzenne et al. Neoplasia 9:479-486 (2007), the entire content of which is hereby incorporated by reference in its entirety.)

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A method, of treating a skin abnormality in a mammalian subject, comprising:
introducing, into a vasculature of the subject, red blood cells (RBCs) that comprise a photosensitive compound; and then
permitting to pass a time-period sufficient for some of the RBCs to enter a region of the subject that comprises the skin abnormality; and then
exposing RBCs in the region to an amount of radiation energy sufficient to result in the photosensitive compound mediating a hyperthermic therapy, a thermal therapy, an oxygen singlet therapy, a radical molecule therapy, or a combination thereof on the skin abnormality,
wherein the photosensitive compound comprises a dye and is substantially encapsulated within the RBCs, and wherein the radiation energy consists essentially of radiation wavelengths absorbed substantially more efficiently by the photosensitive compound than by an epidermal tissue of the subject.

2. The method of claim 2, wherein the skin abnormality comprises a port wine stain, a birthmark, a hemangioma, or a melanoma, and wherein the dye comprises an indocyanine green (ICG), and wherein one or more of the radiation wavelengths are between about 650 nm and about 900 nm.

3. The method of claim 2, wherein the radiation wavelengths are generated by an intense pulsed light (IPL) device.
